(19) United States
(12) Patent Application Publication
(10) Pub. No.: US 2021/0386906 A1
(43) Pub. Date: Dec. 16, 2021

(54) METHODS AND FORMULATIONS FOR TRANSDERMAL ADMINISTRATION OF DERMAL CONTOURING AGENTS

(71) Applicant: DYVE BIOSCIENCES, INC., Thousand Oaks, CA (US)

(72) Inventors: Ryan Beal, Thousand Oaks, CA (US); Luke Gonzales, Thousand Oaks, CA (US); Kilmar Martinez, Thousand Oaks, CA (US); Brandon Sand, Thousand Oaks, CA (US); Lisa Misell, Thousand Oaks, CA (US); Nathan Fitzsimmons, Thousand Oaks, CA (US)

(73) Assignee: DYVE BIOSCIENCES, INC., Thousand Oaks, CA (US)

(21) Appl. No.: 17/288,487

(22) PCT Filed: Oct. 23, 2019
(86) PCT No.: PCT/US2019/057730

§ 371 Date: Apr. 23, 2021

Related U.S. Application Data
(60) Provisional application No. 62/749,556, filed on Oct. 23, 2018.

(51) Int. Cl. A61L 27/20; A61L 27/02; A61L 27/52; A61L 27/18

(52) U.S. Cl. CPC A61L 27/20; A61L 27/025; A61L 2400/06; A61L 27/18; A61L 27/52

(57) ABSTRACT

Disclosed herein is a pre-treatment and/or treatment formulation for transdermal delivery of a dermal pre-treatment agent and/or one or more dermal contouring agents through the skin of a subject, wherein the pre-treatment formulation comprises: a) a buffering agent comprising at least one carbonate salt, lysine, tris, a phosphate buffer and/or 2-imidazole-1-yl-3-ethoxycarbonylpropionic acid (IEPA), or a combination thereof in an amount between about 0.05-60% w/w; and b) a pre-treatment penetrant portion in an amount between about 40 to 99.95% w/w, and wherein the treatment formulation comprises: a) one or more acylating agents in an amount between about 0.25-25% w/w; and b) a treatment penetrant portion in an amount between about 40 to 75% w/w and methods for transdermal delivery of the pretreatment and/or treatment formulation through the skin of a subject.

### BACKGROUND

The subject of this patent application relates generally to topical treatments that effect the dermal contour of the skin. More particularly, it concerns direct application of a penetrating formulation containing a pre-treatment and/or treatment to a subject.

Skin contains a large number of different morphological structures that are composed of various extracellular matrix (ECM) components. Inherent in some conditions, such as localized lipodystrophy (cellulite) and following injury and destruction, ECM restoration has to be achieved by a controlled de novo synthesis or by means of an extrinsic process to restore the native structural integrity of the tissue.

Solar and intrinsic aging both result in the loss of the youthful structural tone of our skin allowing it to deform under its own weight due to the constant force of gravity. The visible signs of aging are fine lines, wrinkles, furrows and skin laxity. The fibrous proteins, responsible for the structural integrity of our skin, are age-depleted by the up-regulation of three matrix metalloproteinases (MMPs), collagenase, a 92-kD gelatinase and stromelysin. It takes but a single exposure to ultraviolet irradiation to induce the degradation of the most abundant structural protein in the body, collagen.

Dermal damage induced by UV irradiation is principally manifested histologically as the disorganization of collagen fibrils and the accumulation of abnormal elastin-containing material. Biochemical evidence of connective tissue alterations in photodamaged skin includes reduced levels of types I and III collagen precursors and cross-links, an increased ratio of type III to type I collagen, and an increased level of elastin.

In attempts to restore the skin laxity manifested by wrinkles and furrows, dermal fillers were introduced to the U.S. market in 1981. Since the introduction of the first dermal bovine collagen-based fillers, the practice of minimally invasive facial rejuvenation using these products has grown exponentially.

The term “dermal filler” does not capture the breath of products that have emerged to volumize and contour the skin of the face due to the deficits from facial fat loss, fat movement and skeletal remodeling.

Advances in technology and application techniques, as well as the development of a better understanding of patients' aesthetic needs have led practitioners to think differently about fillers. It has been stated that the ideal filler would be composed of an easily injectable hypoallergenic substance that could be naturally incorporated into a patients' tissue without risk for adverse reactions. Additionally, the ideal filler would be inexpensive, painless and provide consistent and long-lasting, yet reversible results.

So far, no product has been introduced to satisfy all of the ideal filler criteria, however, great strides have been made since the first bovine collagen-based fillers of the 1980s. First approved by the FDA in 2003, hyaluronic acid (HA) fillers fulfill a number of properties of the ideal filler where collagen products once fell short. Because HA is homologous across species, the need for allergy testing is obviated. HA filler produce results that last for 6 to 9 months or longer and the effects can be reversed with hyaluronidase in the event of unwanted results.

HA fillers are the most frequently injected dermal fillers in the United States. The molecular structure of the HA monomer is identical across all HA fillers. When injected into human tissue, the raw HA monomer is quickly broken down in the enzymatic degradation of endogenous hyaluronidase; to overcome this response, manufacturers add cross-linkers to un-cross-linked HA gel to create an HA gel that imposes both a physical and chemical barrier to enzymatic breakdown. A greater degree of cross-linking corresponds to a harder gel and more longevity in the treated tissue, however, as the gel becomes harder and more cohesive, more force is required during injection and the possibility of eliciting an immune response is increased.

There are a variety of other filler materials that currently are FDA approved, including calcium hydroxylapatite (Radiesse, Merz Aesthetics, Inc.), poly-L-lactic acid (Sculptra, Galderma Aesthetics), and polymethylmethacrylate (Artefill, Suneva Medical, Inc.).

With the growing popularity of dermal fillers, there continues to be a demand for a more ideal filler. Volume deficits from facial fat loss, fat movement and skeletal remodeling are becoming increasingly recognized as integral contributors to facial aging. Accordingly, a trend towards a more volumizing filler, that can replace these deficits and recontour the face, has emerged.

Interestingly, a lifting effect may be achieved by three dimensional (3-D) volumizing the midface, resulting in improvement of the midface and nasolabial folds.

The formulation of this invention allows for the safe and effective delivering of acylation agents to intact tissue. Studies of the topical application of this novel composition and method have revealed the expeditious increase in the three-dimensional (3-D) volume of the targeted tissue site. No adverse events have been observed and this invention would appear to satisfy most, in not all, of the criteria for an ideal dermal filler and tissue volumizer.

Applicant hereby incorporates herein by reference any and all patents and published patent applications cited or referred to in this application. Aspects of the present invention fulfill these needs and provide further related advantages as described in the following summary.

### SUMMARY

Aspects of the present invention teach certain benefits in construction and use which give rise to the exemplary advantages described below.

The present invention solves the problems described above by providing buffering formulations with improved penetration. In at least one embodiment, disclosed herein are formulations containing carbonate salts useful in conditions where buffering therapy is needed.

In one aspect, disclosed herein is a pre-treatment and/or treatment formulation for transdermal delivery of a dermal pre-treatment agent and/or one or more dermal contouring agents through the skin of a subject, wherein the pre-treatment formulation comprises: a) a buffering agent comprising at least one carbonate salt, lysine, tris, a phosphate buffer and/or 2-imidazole-1-yl-3-ethoxycarbonylpropionic acid (TEPA), or a combination thereof in an amount between about 0.05-60% w/w; and b) a pre-treatment penetrant portion in an amount between about 40 to 99.95% w/w, and wherein the treatment formulation comprises: a) one or more acylating agents in an amount between about 0.25-25% w/w; and b) a treatment penetrant portion in an amount between about 40 to 75% w/w.

In another aspect, disclosed herein is a method for transdermal delivery of one or more buffering agents through the skin of a subject, comprising: a pre-treatment and/or treatment formulation for transdermal delivery of a dermal pre-treatment agent and/or one or more dermal contouring agents through the skin of a subject, wherein the pre-treatment formulation comprises: a) a buffering agent comprising at least one carbonate salt, lysine, tris, a phosphate buffer and/or 2-imidazole-1-yl-3-ethoxycarbonylpropionic acid (TEPA), or a combination thereof in an amount between about 0.05-60% w/w; and b) a pre-treatment penetrant portion in an amount between about 40 to 99.95% w/w, and wherein the treatment formulation comprises: a) one or more acylating agents in an amount between about 0.25-25% w/w; and b) a treatment penetrant portion in an amount between about 40 to 75% w/w.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

FIG. 1 shows the effectiveness of the formulations of the invention in removing crow's feet in a subject. FIG. 1A shows the subject before treatment and FIG. 1B shows the subject 15 minutes after treatment.

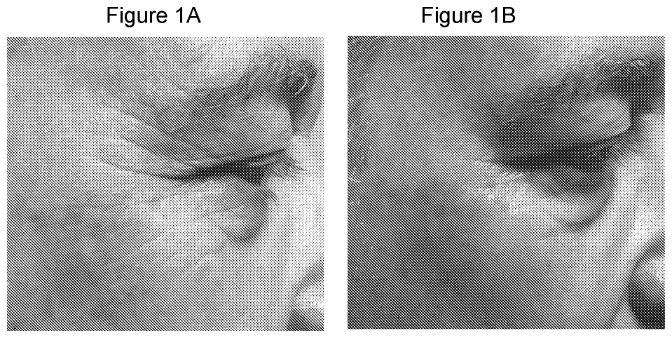

FIG. 2 shows the sustained effect of this treatment where the subject shown in FIG. 1A is shown again in FIG. 2A and in FIG. 2B the same subject is shown approximately four months after treatment.

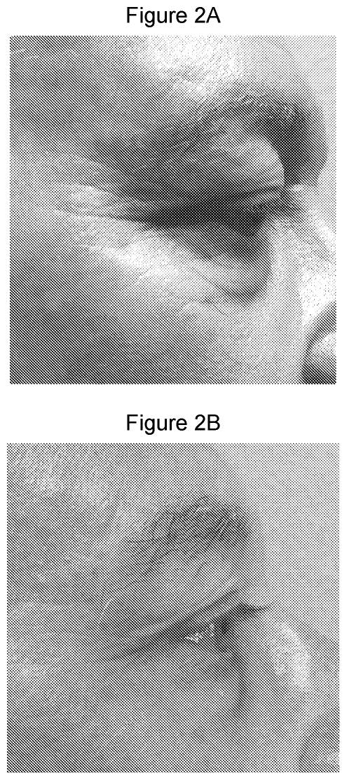

FIG. 3 shows treatment of another subject for crow's feet using the formulations of the invention. FIG. 3A shows the subject prior to treatment and FIG. 3B shows the subject 15 minutes after treatment.

### DETAILED DESCRIPTION

In one aspect, disclosed herein is a formulation for transdermal delivery of one or more buffering agents through the skin of a subject, comprising: a buffering agent comprising at least one carbonate salt, lysine, tris, a phosphate buffer and/or 2-imidazole-1-yl-3-ethoxycarbonylpropionic acid (TEPA), or a combination thereof in an amount between about 10-56% w/w; and a penetrant portion in an amount between about 44 to 90% w/w, wherein the penetrant portion comprises water in an amount less than about 85% w/w, and wherein the formulation comprises less than about 12% w/w lecithin.

In another aspect, disclosed herein is a method for transdermal delivery of one or more buffering agents through the skin of a subject, comprising: a buffering agent comprising at least one carbonate salt, lysine, tris, a phosphate buffer and/or 2-imidazole-1-yl-3-ethoxycarbonylpropionic acid (TEPA), or a combination thereof in an amount between about 10-56% w/w; and a penetrant portion in an amount between about 44 to 90% w/, wherein the penetrant portion comprises water in an amount less than about 85% w/w, and wherein the formulation comprises less than about 12% w/w lecithin.

As skin ages, there is a natural process whereby collagen and elastin breakdown in the skin, primarily driven by UV exposure. This process results in multiple changes in the appearance of the skin including fine lines and wrinkles, skin laxity, generalized creepiness and thinning of the skin. For example, fine lines emerge in the late 20s/early 30s which deepen as the skin ages and thinning of the skin contributes to the appearance of cellulite. Aesthetic treatments to prevent and correct lines and wrinkles are a significant proportion of all aesthetic procedures and represent 7-10M treatments in the US alone and over 80% of post-adolescent females experience posterolateral thigh cellulite.

This disclosure herein embodies a way to increase the thickness and alter the biochemical characteristics of the skin. These changes lead to the improved appearance of the skin changes characteristic of aging. The invention exploits the inherent aqueous instability of all anhydrides, sulfonyl chlorides, acid chlorides and sulfonic acids. They rapidly react with water and undergo hydrolysis into an acid form (inactive) of the compound. Acylating agents rapidly react with free amines on isolated proteins and proteins in tissue, converting the free amine to another chemical structure inherent in the specific acylation chemical. For example, treatment of deprotonated free amines with glutaric anhydride converts the NH₃⁺ to COO⁻ (negative charge). Treatment with acetic anhydride converts NH₃⁺ to CH₃ (neutral charge). Previous studies have demonstrated that treatment of human dermis with glutaric anhydride increases thickness and changes dermal biomechanical characteristics.

This disclosure herein provides for a method of safe and effective topical delivery acylation agents to alter net charge and net charge density of the treated tissue. The invention results in an increase in the net negative charge on reactive tissue proteins, thereby increasing water binding characteristics of selectively treated tissue. This process will result in the selective swelling of the targeted site, thereby, increasing the targeted dermal thickness and pliability.

### Existing Treatments

Bototulinium toxin injections for dimunition of muscle activity causing wrinkles, hyaluronic acid and other fillers to volumize wrinkles, various light-based medical devices aimed at stimulating collagen production, surgical tightening, etc. There is a lack of effective topical treatments that achieve the desired effect.

Botox (botulinum toxin) injections (and other similar toxins) can be very effective, but require painful injections, lead to muscle diminution (especially facial), and can take 4-7 days to take effect.

Fillers, another common approach, require careful targeted injections and are thus vulnerable to physician errors and are not as convenient for use in finer lines.

In an academic setting, glutaric anhydride (GA) has been used on in vitro bare collagen and shown to induce hygroscopy by reacting with collagen fibers and increasing their affinity for binding water molecules. It has been used on a limited basis in vivo on human subjects by applying acylation agents to skin in which the stratum corneum has been damaged or breached by means of abrasion, tape-stripping, microneedling, or injection.

Challenges with Transdermal Treatments:

Current topical treatments either require mechanical means (e.g., needles) or damage to the stratum corneum. In addition, the results achieved with alternate methods have not proven to be

The transdermal formulations disclosure herein allows for the quick and efficacious topical deliver with immediate and lasting aesthetic results without the need for mechanically altering the stratum corneum. It involves a two-step application whereby the first deprotonates free amines on collagen molecules and the second stage allows the delivery of acylating agents that bind to the free amines and thereby increase the water binding characteristics of collagen. The increased water binding results in selective and long-lasting swelling of the targeted site, thereby, increasing the targeted dermal thickness and pliability.

### Step One—Anhydrous Buffering Treatment

We have developed a pre-treatment skin prep step that de-protonates lysine residues in the collagen to increase the effectiveness of the Acylation Step.

### Step Two—Acylation Agent Treatment

We have developed a formulation that allows for transdermal delivery of GA.

In some embodiments, the order of the steps may be reversed or repeated as needed.

By way of example, there are a number of additional agents that could be used in a manner similar to glutaric anhydride. In some embodiments, the acylating agent is selected from the group consisting of glutaric anhydride, EDTA anhydride, octenyl-succinic anhydride, maleic anhydride, succinic anhydride, citraconic anhydride, methyl succinic anhydride, itaconic anhydride, methyl glutaric anhydride, dimethyl glutaric anhydride, phthalic anhydride, oxalyl chloride, malonyl chloride, (chlorosulfonyl)acetylchloride, (chlorosulfonyl)benzoic acid, 4-chloro-3-(chlorosulfonyl)-5-nitroebnzoicacid, 3-(chlorosulfonyl)-p-anisic acid, 3-(sulfonyl)benzoic acid, 3,5-Dimethoxybenzoyl chloride, acetic anhydride, chloroacetic anhydride, propionic anhydride, butyric anhydride, isobutyric anhydride, isovaleric anhydride, hexanoic anhydride, acetylchloride, propionylchloride, dichloropropionylchloride, butyryl chloride, isobutyryl chloride, valeryl chloride, ethanesulfonyl chloride, methanesulfonyl chloride, 1-butanesulfonyl chloride, 4,6-diamino-2-methylthiopyrimidine-5-Sulfonic acid, and combinations thereof.

In some embodiments, the alkylating agent is an amount between about 0.25-25% w/w. In some embodiments, the alkylating agent is an amount between about 1.5-25% w/w. In some embodiments, the alkylating agent is an amount between about 1.5-15% w/w. In some embodiments, the alkylating agent is an amount between about 1.5-10% w/w. In some embodiments, the alkylating agent is an amount between about 1.5-5% w/w. In some embodiments, the alkylating agent is an amount of about 4% w/w. Disclosed herein is an optimized dosing (i.e., percent active of glutaric anhydride). Experimental test results indicated a range of alkylating agent of between about 1.5-5.0 w/w % and that the best efficacy was achieved in an amount of between about 1.5-4.0 w/w %.

Disclosed herein is an optimized dose timing for both the pre-treatment and active steps. For the initial pre-treatment step, we tested treatment times between 10-40 minutes, and showed that the optimum time was 20 minutes on the skin. There were notable improvements at that timing above 10-15 minute tests, but the efficacy benefit plateaued at 20 minutes, and we even saw some minor damage to the skin when the pre-treatment was left on 30+ minutes. For the GA treatment, after testing the same 10-40 minute range, 30 minutes was shown to be the optimum timing where efficacy plateaued and there were not any signs of skin damage.

In some embodiments, the formulation employs penetrants described US2009/0053290 ('290), WO2014/209910 ('910), and WO2017/127834. The present formulations may include a nonionic surfactant.

Briefly, the penetrants described in the above-referenced US and PCT applications are based on combinations of synergistically acting components. Many such penetrants are based on combinations of an alcohol, such as benzyl alcohol to provide a concentration of 0.5-20% w/w of the final formulation with lecithin organogel present in the penetrant to provide 25-70% w/w of the formulation. These penetrants are also useful when the agent is a buffer, such as sodium bicarbonate, but less lecithin organogel may be required—e.g. less than 12% w/w when the sodium bicarbonate is present at high concentration as disclosed herein.

In some embodiments, the buffering agent is any mildly basic compound or combination that will result in a pH of 7-8 in the microenvironment of the tumor cells. In some embodiments, the formulation has a pH of 7-10. Such buffers, in addition to carbonate and/or bicarbonate salts, include lysine buffers, chloroacetate buffers, tris buffers (i.e., buffers employing tris (hydroxymethyl) aminoethane), phosphate buffers and buffers employing non-natural amino acids with similar pKa values to lysine, and/or 2-imidazole-1-yl-3-ethoxycarbonylpropionic acid (TEPA). For example, the enantiomers of native forms of such amino acids or analogs of lysine with longer or shorter carbon chains or branched forms thereof. Histidine buffers may also be used. Typically, the concentration of buffer in the pre-treatment formulation is in the range of 1-56% w/w or 10-36% w/w. In some embodiments, the pre-treatment formulation has carbonate salt is in an amount between about 1-36% w/w of the formulation. More typical ranges for the carbonate salt with the pre-treatment formulation are 10-36% by weight. However, the upper limits in terms of skin irritation for sodium carbonate is an amount greater than about 7.0% w/w. Moreover, Group II carbonate salts have limited solubility and would not dissociate upon contact with skin of a subject.

Alternatively, the penetrant component comprises a completion component as well as buffer agent in sufficient quantity to impart viscosity and viscoelasticity, one or more surfactants and an alcohol. The completion component can be a polar liquid, a non-polar liquid or an amphiphilic substance.

In some embodiments, the carbonate salt is sodium carbonate and/or sodium bicarbonate milled to a particle size is less than 200 μm. In some embodiments, the carbonate salt is sodium carbonate and/or sodium bicarbonate milled to a particle size is less than 70 μm. In some embodiments, the carbonate salt is sodium carbonate and/or sodium bicarbonate milled to a particle size is less than 70 μm, wherein the sodium bicarbonate is solubilized in the formulation in an amount less than about 10% w/w of the formulation. In some embodiments, the sodium bicarbonate is milled to a particle size is less than 70 μm, less than 1 μm, less than 500 nm, less than 100 nm, or less than 50 nm, wherein particle sizes less than about 10 μm have an enhanced penetration thru the skin of a subject. In some embodiments, the sodium bicarbonate is jet milled to a particle size less than about 70 μm. In some embodiments, the sodium bicarbonate is Sodium Bicarbonate USP Grade 3DF that has a particle size distribution less than 70 μm.

In some embodiments, the one or more pre-treatment and/or treatment formulations are formulated with Aveeno® moisturizers, cream, oils, lotions; Jergens® moisturizers, cream, oils, lotions; Honest Company® moisturizers, cream, oils, lotions; Dermologica® moisturizers, cream, oils, lotions; or St. Ives™ moisturizers, cream, oils, lotions. In some embodiments, the commercial lotions, moisturizers, etc. are formulated with the buffering agent comprising a carbonate salt, lysine, tris, a phosphate buffer and/or 2-imidazole-1-yl-3-ethoxycarbonylpropionic acid (TEPA) in an amount between about 10-56% w/w.

The penetrant portion is a multi-component mixture, whereby the particular concentrations of the penetration enhancers are informed in part by the particle size of the sodium bicarbonate. The formulation enables the sodium bicarbonate to become bio-available to the target site within minutes of topical administration. The formulations permit the use of minimal concentrations of therapeutic agents, as little as. 1/1000th of concentrations required of alternative processes, while enabling bioactivity and positive clinical outcomes simultaneously. In some embodiments, the penetrant portion comprises an alcohol in an amount less than 5% w/w of the formulation.

Subjects of the disclosure herein, in addition to humans, include veterinary subjects, wherein formulations suitable for these subjects are also appropriate. Such subjects include livestock and pets as well as sports animals such as horses and greyhounds.

The formulations comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

For topical administration, and in particular transdermal administration, the formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. Particularly suitable penetrants especially for those that contain at least one agent other than buffer include those that are described in the above-referenced US2009/0053290 ('290), WO2014/209910 (910), and WO2017/127834. In addition to formulations with penetrants, transdermal delivery can be effected by mechanically disrupting the surface of the skin to encourage penetration, or simply by supplying the formulation applied to the skin under an occlusive patch.

Alternatively, the penetrant portion comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Lecithin organogel is a combination of lecithin with a gelling component. Suitable gelling components also include isopropyl palmitate, ethyl laurate, ethyl myristate and isopropyl myristate. In some embodiments, the formulation comprises a gelling agent in an amount less than 5% w/w of the formulation. Certain hydrocarbons, such as cyclopentane, cyclooctane, trans-decalin, trans-pinane, n-pentane, n-hexane, n-hexadecane may also be used. Thus, an important permeation agent is a lecithin organogel, wherein the combination resulting from lecithin and the organic solvent acts as a permeation agent. In some embodiments, the formulation comprises less than about 7% w/w or less than about 12% w/w lecithin. In some embodiments, the penetrant portion comprises lecithin organogel, an alcohol, a surfactant, and a polar solvent. In some embodiments, the lecithin organogel is a combination of soy lecithin and isopropyl palmitate. In some embodiments, the penetrant portion comprises lecithin and isopropyl palmitate, undecane, isododecane, isopropyl stearate, or a combination thereof. In some embodiments, the formulation comprises Lipmax™ in an amount between about 1-20% w/w or an equivalent 50/50 mixture of isopropyl palmitate and lecithin. Lecithin organogels are not always clear or thermodynamically stable, but are viscoelastic, and biocompatible phases composed of phospholipids and appropriate organic liquid. An example of a suitable lecithin organogel is lecithin isopropyl palmitate, which is formed when isopropyl palmitate is used to dissolve lecithin. The ratio of lecithin to isopropyl palmitate may be 50:50. Illustrated below in the Examples is a formulation containing soy lecithin in combination with isopropyl palmitate; however, other lecithins could also be used such as egg lecithin or synthetic lecithins. Various esters of long chain fatty acids may also be included. Methods for making such lecithin organogels are well known in the art. In most embodiments, the lecithin organogel is present in the final formulation is less than about 20% w/w. In some embodiments, the concentration of lecithin organogel may be as low as 0.5% w/w, 1% w/w, 5% w/w, 10% w/w or 20% w/w. In some embodiments, the penetrant portion comprises a mixture of xanthan gum, lecithin, sclerotium gum, pullulan, or a combination thereof in an amount less than 2% w/w, 5% w/w, or 10% w/w of the formulation. In some embodiments, the formulation comprises Siligel™ in an amount between about 1-5% w/w or 5-15% w/w, or an equivalent mixture of xanthan gum, lecithin, sclerotium gum, and pullulan. In some embodiments, the penetrant portion comprises a mixture of caprylic triglycerides and capric triglycerides in amount less than 2% w/w, 8% w/w, or 10% w/w of the formulation. In some embodiments, the formulation comprises Myritol® 312 in an amount between about 0.5-10% w/w, or an equivalent mixture of caprylic triglycerides and capric triglycerides.

In some embodiments, the penetrant portion is in an amount between about 44-90% w/w or 44-80% w/w of the formulation. In some embodiments, the penetrant portion comprises phosphatidyl choline in amount less than 7% w/w, less than 12% w/w, or 18% w/w of the formulation. In some embodiments, the penetrant portion comprises a phospholipid in amount less than 12% w/w or 18% w/w of the formulation. In some embodiments, the penetrant portion comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the penetrant portion comprises cetyl alcohol in amount less than 2% w/w, 5% w/w, or 8% w/w of the formulation. In some embodiments, the penetrant portion comprises benzyl alcohol in an amount less than about 2% w/w, 5% w/w, or 8% w/w. In some embodiments, the penetrant portion comprises stearic acid in an amount less than 2% w/w, 5% w/w, or 8% w/w of the formulation. In some embodiments, the penetrant portion comprises water in an amount between about 0-85% w/w. In some embodiments, the penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 12% w/w or in amount less than 7% w/w of the formulation.

Lecithin organogels may be in the form of vesicles, microemulsions and micellar systems. In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semi-solid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine.

The lecithin organogels are formulated with an additional component to assist in the formation of micelles or vascular structures. In one approach, the organogels are formulated with a polar component such as water, glycerol, ethyleneglycol or formamide, in particular with water. In general, a nonionic detergent such as a poloxamer in aqueous solution is used to top off. Certain detergents, such as Tween® 80 or Span® 80 may be used as alternatives. The percentage of these components in the anhydrous forms of the composition is in the range of 1-15% w/w. In these essentially anhydrous forms, powdered or micronized nonionic detergent is used to top off, typically in amounts of 1-30% w/w of the penetrant portion. In one approach to determine the amount of bile salt, the % is calculated by dividing the % w/w of lecithin by 10.

An additional component in the formulations of the disclosure is an alcohol. Benzyl alcohol and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are included. Due to the aromatic group present in a permeation enhancement formulation such as benzyl alcohol, the molecule has a polar end (the alcohol end) and a non-polar end (the benzene end). This enables the agent to dissolve a wider variety of drugs and agents. The alcohol concentration is substantially lower than the concentration of the lecithin organogel in the composition.

In some embodiments, as noted above, the performance of the formulations is further improved by including a nonionic detergent and polar gelling agent or including a powdered surfactant. In both aqueous and anhydrous forms of the composition, detergents, typically nonionic detergents are added. In general, the nonionic detergent should be present in an amount between about 1% w/w to 30% w/w of the penetrant portion. Typically, in the compositions wherein the formulation is topped off with a polar or aqueous solution containing detergent, the amount of detergent is relatively low—e.g., 2-25% w/w, or 5-15% w/w or 7-12% w/w of the penetrant portion. However, in compositions that are essentially anhydrous and are topped-off by powdered detergent, relatively higher percentages are usually used—e.g., 20-60% w/w.

In some embodiments, the penetrant portion further comprises a detergent portion in an amount between about 1 to 70% w/w or 1-60% w/w of the penetrant portion. In some embodiments, the nonionic detergent provides suitable handling properties whereby the formulations are gel-like or creams at room temperature. To exert this effect, the detergent, typically a poloxamer, is present in an amount between about 2-12% w/w of the penetrant portion, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, the formulation also contains surfactant, typically, nonionic surfactant at 2-25% w/w of the penetrant portion along with a polar solvent wherein the polar solvent is present in an amount at least in molar excess of the nonionic surfactant. In these embodiments, typically, the composition comprises the above-referenced amounts of lecithin organogel and benzyl alcohol along with a carbonate salt with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3-15% w/w, and again includes intermediate percentages such as 5% w/w, 7% w/w, 10% w/w, 12% w/w, and the like. In some embodiments, the detergent portion comprises a nonionic surfactant in an amount between about 1-30% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethyleneglycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Typical components that may also be included in the formulations are fatty acids, terpenes, lipids, and cationic, and anionic detergents. In some embodiments, the formulation further comprises tranexamic acid in an amount less than 2% w/w, 5% w/w, or 10% w/w of the formulation. In some embodiments, the formulation further comprises a polar solvent in an amount less than 2% w/w, 5% w/w, 10% w/w, or 20% w/w of the formulation. In some embodiments, the formulation further comprises a humectant, an emulsifier, an emollient, or a combination thereof. In some embodiments, the formulation further comprises almond oil in an amount less than about 5% w/w. In some embodiments, the formulation further comprises a mixture of thermoplastic polyurethane and polycarbonate in an amount less than about 5 w/w. In some embodiments, the formulation further comprises phosphatidylethanolamine in an amount less than about 5% w/w. In some embodiments, the formulation further comprises an inositol phosphatide in an amount less than about 5% w/w.

Other solvents and related compounds that may be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include α-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that may be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants may be included, such as vitamin C, vitamin E, proanthocyanidin and α-lipoic acid typically in concentrations of 0.1%-2.5% w/w.

Other agents include inhibitors of E-cadherin and of epidermal growth factor receptor (EGFR). Known inhibitors include erlotinib, an anti-integrin drug (Cilengitide), Cariporide, Eniporide and Amiloride.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of the formulation itself. The application of the formulation may be by simple massaging onto the skin or by use of devices such as syringes or pumps. Patches could also be used. In some cases, it is helpful to cover the area of application to prevent evaporation or loss of the formulation.

Where the application area is essentially skin, it is helpful to seal-off the area of application subsequent to supplying the formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In one aspect, disclosed herein is a pre-treatment and/or treatment formulation for transdermal delivery of a dermal pre-treatment agent and/or one or more dermal contouring agents through the skin of a subject,

wherein the pre-treatment formulation comprises:
a) a buffering agent comprising at least one carbonate salt, lysine, tris, a phosphate buffer and/or 2-imidazole-1-yl-3-ethoxycarbonylpropionic acid (TEPA), or a combination thereof in an amount between about 0.05-60% w/w; and
b) a pre-treatment penetrant portion in an amount between about 40 to 99.95% w/w, and

wherein the treatment formulation comprises:
a) one or more acylating agents in an amount between about 0.25-25% w/w; and
b) a treatment penetrant portion in an amount between about 40 to 75% w/w.

In some embodiments, the acylating agent is selected from the group consisting of glutaric anhydride, EDTA anhydride, octenyl-succinic anhydride, maleic anhydride, succinic anhydride, citraconic anhydride, methyl succinic anhydride, itaconic anhydride, methyl glutaric anhydride, dimethyl glutaric anhydride, phthalic anhydride, oxalyl chloride, malonyl chloride, (chlorosulfonyl)acetylchloride, (chlorosulfonyl)benzoic acid, 4-chloro-3-(chlorosulfonyl)-5-nitroebnzoicacid, 3-(chlorosulfonyl)-p-anisic acid, 3-(sulfonyl)benzoic acid, 3,5-Dimethoxybenzoyl chloride, acetic anhydride, chloroacetic anhydride, propionic anhydride, butyric anhydride, isobutyric anhydride, isovaleric anhydride, hexanoic anhydride, acetylchloride, propionylchloride, dichloropropionylchloride, butyryl chloride, isobutyryl chloride, valeryl chloride, ethanesulfonyl chloride, methanesulfonyl chloride, 1-butanesulfonyl chloride, 4,6-diamino-2-methylthiopyrimidine-5-Sulfonic acid, and combinations thereof.

In some embodiments, the pre-treatment formulation is anhydrous.

In some embodiments, the pre-treatment and/or treatment formulation comprises less than about 12% w/w lecithin.

In some embodiments, the pre-treatment and/or treatment penetrant portion further comprises a detergent portion in an amount between about 1 to 70% w/w.

In some embodiments, the detergent portion comprises a nonionic surfactant in an amount between about 2-25% w/w of the penetrant portion; and a polar solvent in an amount less than 5% w/w of the penetrant portion.

In some embodiments, the buffering agent is in an amount between about 0.05-36% w/w of the formulation.

In some embodiments, the pre-treatment and/or treatment penetrant portion is in an amount between about 44-80% w/w of the formulation.

In some embodiments, the pre-treatment and/or treatment penetrant portion comprises an alcohol in an amount less than 10% w/w of the formulation.

In some embodiments, the pre-treatment and/or treatment penetrant portion comprises lecithin organogel, an alcohol, a surfactant, and a polar solvent.

In some embodiments, the pre-treatment and/or treatment penetrant portion comprises a mixture of xanthan gum, lecithin, sclerotium gum, pullulan, or a combination thereof in an amount less than 5% w/w of the formulation.

In some embodiments, the pre-treatment and/or treatment penetrant portion comprises a mixture of caprylic triglycerides and capric triglycerides in amount less than 8% w/w of the formulation.

In some embodiments, the pre-treatment and/or treatment penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 12% w/w of the formulation.

In some embodiments, the pre-treatment and/or treatment penetrant portion comprises cetyl alcohol in amount less than 5% w/w of the formulation.

In some embodiments, the pre-treatment and/or treatment penetrant portion comprises stearic acid in an amount less than 5% w/w of the formulation.

In some embodiments, the pre-treatment and/or treatment formulation comprises a gelling agent in an amount less than 5% w/w of the formulation.

In some embodiments, the carbonate salt is sodium bicarbonate milled to a particle size less than 70 μm, wherein the sodium bicarbonate is solubilized in the formulation in an amount less than 10% w/w of the formulation.

In some embodiments, the pre-treatment and/or treatment formulation each independently further comprise tranexamic acid in an amount less than 5% w/w of the formulation.

In some embodiments, the pre-treatment and/or treatment formulation each independently further comprise a polar solvent in an amount less than 5% w/w of the formulation.

In some embodiments, the pre-treatment and/or treatment formulation each independently further comprise a humectant, an emulsifier, an emollient, or a combination thereof.

In some embodiments, the pre-treatment formulation has a pH of 7-10.5.

In some embodiments, disclosed herein is a formulation for transdermal delivery of a buffering agent through the skin of a subject, comprising a formulation of Table 1, Table 2, Table 3, Table 4, Table 5, Table 6, Table 7, Table 8, Table 9, Table 10, Table 11, Table 12, Table 13, or Table 14.

In another aspect, disclosed herein is a method for transdermal delivery of the pre-treatment and/or treatment formulation of claim 1, through the skin of a subject.

In some embodiments, the pre-treatment formulation is applied to a subject first and the treatment formulation is applied to the subject after the pre-treatment formulation.

In another aspect, disclosed herein is a method to expand tissue volume comprising applying the pre-treatment and/or treatment formulation of claim 1, through the skin of a subject, the method comprising: a) mixing the one or more acylating agents in powdered form with the treatment penetration portioning immediately followed by application of the mixture to a skin area of the subject.

In another aspect, disclosed herein is a method to expand tissue volume comprising applying the pre-treatment and/or treatment formulation of claim 1, through the skin of a subject, the method comprising: a) mixing the one or more acylating agents in powdered form with the treatment penetration portioning immediately followed by application of the mixture to a skin area of the subject; or and first, applying to a skin area of the subject the pre-treatment formulation buffered at pH 7-11.5 comprising an effective amount of said acylating agent, 25-70% w/w lecithin organogel, 1-20% w/w benzyl alcohol, and a nonionic detergent in the presence of a bile salt; and second mixing the one or more acylating agents in powdered form with the treatment penetration portioning immediately followed by application of the mixture to a skin area of the subject.

In another aspect, disclosed herein is a method to expand volume and maintain hydration of tissue which method comprises applying to an area the pre-treatment and/or treatment formulation of claim 1, through the skin of a subject, the method comprising: a) mixing the one or more acylating agents in powdered form with the treatment penetration portioning immediately followed by application of the mixture to a skin area of the subject.

In another aspect, disclosed herein is a method to expand volume and maintain hydration of tissue which method comprises applying to an area the pre-treatment and/or treatment formulation of claim 1, through the skin of a subject, the method comprising: a) mixing the one or more acylating agents in powdered form with the treatment penetration portioning immediately followed by application of the mixture to a skin area of the subject; or b) first, applying to a skin area of the subject the pre-treatment formulation buffered at pH 7-11.5 comprising an effective amount of said acylating agent, 25-70% w/w lecithin organogel, 1-20% w/w benzyl alcohol, and a nonionic detergent in the presence of a bile salt; and second mixing the one or more acylating agents in powdered form with the treatment penetration portioning immediately followed by application of the mixture to a skin area of the subject.

In another aspect, disclosed herein is a method to restore the barrier effect of skin after transdermal treatment, which method comprises applying the pre-treatment and/or treatment formulation of claim 1, through the skin of a subject, the method comprising: a) mixing the one or more acylating agents in powdered form with the treatment penetration portioning immediately followed by application of the mixture to a skin area of the subject

In another aspect, disclosed herein is a method to restore the barrier effect of skin after transdermal treatment, which method comprises applying the pre-treatment and/or treatment formulation of claim 1, through the skin of a subject, the method comprising: a) mixing the one or more acylating agents in powdered form with the treatment penetration portioning immediately followed by application of the mixture to a skin area of the subject; or b) first, applying to a skin area of the subject the pre-treatment formulation buffered at pH 7-11.5 comprising an effective amount of said acylating agent, 25-70% w/w lecithin organogel, 1-20% w/w benzyl alcohol, and a nonionic detergent in the presence of a bile salt; and second mixing the one or more acylating agents in powdered form with the treatment penetration portioning immediately followed by application of the mixture to a skin area of the subject.

In some embodiments, the phrase “through the skin of a subject” refers to through the stratum corneum and epidermis and into the dermis (mid-skin layer) where it acts on the collagen.

In some embodiments, the pre-treatment formulation comprises:
Cetyl alcohol in an amount between about 0.5-10% w/w;
Myritol® 312 in an amount between about 0.5-15% w/w;
Isododecane 1-20% w/w;
Lipmax™ in an amount between about 1-25% w/w;
Phospholipon® 90G in an amount between about 0.5-20% w/w;
Cetiol Ultimate® in an amount less than about 15% w/w;
Tego 13-06 1-10% w/w;
Hyaluronic acid 0.05-10% w/w;
Pluronic® in an amount between about 1-15% w/w;
Propylene glycol in an amount between about 0.5-15% w/w;
Durasoft® in an amount between about 0.1-5% w/w;
Sodium carbonate in an amount between about 1-25% w/w;
Calcium carbonate in an amount between about 1-36% w/w.
Ethanol in an amount between about 0.5-10% w/w; and
Benzyl alcohol in an amount between about 0.25-5% w/w.

In some embodiments, the treatment formulation comprises:
Phospholipon® 90G in an amount between about 0.5-20% w/w;
EDTA (Disodium ethylenediaminetetraacetate dihydrate) in an amount between about 0.05-5% w/w;
Triethanolamine in an amount between about 0.05-2% w/w;
tert-Amyl alcohol in an amount between about 0.5-10% w/w;
Triacetin in an amount between about 43.70-X % w/w;
Isopropyl palmitate in an amount between about 1-10% w/w;
Lipmax™ in an amount between about 1-32% w/w;
Pluronic® in an amount between about 0.1-25% w/w;
Sodium hydroxide in an amount between about 0.005-2% w/w;
Sodium decanoate in an amount between about 0.05-5% w/w;
Sodium bicarbonate in an amount between about 0.05-10% w/w;
Sodium carbonate in an amount between about 0.05-10% w/w;
Benzyl alcohol in an amount between about 0.25-5% w/w; and
Glutaric anhydride in an amount between about X % w/w, wherein X is in an amount between about 1-15% w/w.

In some embodiments, the treatment formulation comprises:
Phospholipon® 90G in an amount between about 0.5-20% w/w;
EDTA (Disodium ethylenediaminetetraacetate dihydrate) in an amount between about 0.05-5% w/w;
Triethanolamine in an amount between about 0.05-2% w/w;
tert-Amyl alcohol in an amount between about 0.5-10% w/w;
Triacetin in an amount between about 1-60% w/w;
Isopropyl palmitate in an amount between about 1-10% w/w;
Lipmax™ in an amount between about 1-32% w/w;
Pluronic® in an amount between about 0.1-25% w/w;
Sodium hydroxide in an amount between about 0.005-2% w/w;
Sodium decanoate in an amount between about 0.05-5% w/w;
Sodium bicarbonate in an amount between about 0.05-10% w/w;
Sodium carbonate in an amount between about 0.05-10% w/w;
Benzyl alcohol in an amount between about 0.25-5% w/w; and
Glutaric anhydride in an amount between about 1-10% w/w

In applying the formulations of the invention, the formulation itself is simply placed on the skin and spread across the surface and/or massaged to aid in penetration. The amount of formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of the formulation to the skin and prevents distortion of the formulation by evaporation in some cases.

The schedule of application is dependent on the nature of the skin treatment being administered. Repeated application is often desirable, for example, during intermittent types of exercise. Alternatively, the formulation may be left in place. Application to supply nutrients to patients may also be for prolonged periods of time.

The composition may be applied to the skin using standard procedures for application such as a brush, a syringe, a gauze pad, a dropper, or any convenient applicator. More complex application methods, including the use of delivery devices, may also be used, but are not required.

### Examples

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof. Ultimately, the formulations may be utilized in virtually any context where buffering therapy with or without a therapeutic agent(s) is desired.

### Example 1: Dose Concentration Optimization for Topically Delivered Glutaric Anhydride to Thicken Skin in an In Vitro Porcine Skin Model

In this experiment, glutaric anhydride at different concentrations in formulations of the invention in combination with a pre-treatment anhydrous buffering step in formulations of the invention was tested for its ability to thicken skin in an in vitro porcine skin model.

In vitro tests were performed as follows: Porcine skin samples had their thickness measured, then were randomly sorted into treatment groups and given their respective treatments (described below). Trial design was built to assess these primary outcomes: change in skin thickness. Treatment groups randomized as follows:

a. Control—no treatment
b. 20 minutes of Anhydrous Buffering Treatment (Table 1) followed by 30 minutes of Acylation Agent Treatment (Table 2) with Glutaric Anhydride concentration of 1.67%
c. 20 minutes of Anhydrous Buffering Treatment (Table 1) followed by 30 minutes of Acylation Agent Treatment (Table 2) with Glutaric Anhydride concentration of 3.33%
d. 20 minutes of Anhydrous Buffering Treatment (Table 1) followed by 30 minutes of Acylation Agent Treatment (Table 2) with Glutaric Anhydride concentration of 5.00%
e. 20 minutes of Anhydrous Buffering Treatment (Table 1) followed by 30 minutes of Acylation Agent Treatment (Table 2) with Glutaric Anhydride concentration of 6.67%
f. 20 minutes of Anhydrous Buffering Treatment (Table 1) followed by 30 minutes of Acylation Agent Treatment (Table 2) with Glutaric Anhydride concentration of 10.00%

TABLE 1
Anhydrous Buffering Treatment Formulation

| Chemical | |
|---|---|
| Component | Wt % |
| cetyl OH | 2.00% |
| Myritol ® 312 | 8.00% |
| isododecane | 9.90% |
| Lipmax ™ | 4.00% |
| phospholipon 90G | 9.00% |
| Cetiol ® Ultimate | 7.00% |
| Tego ® 13-06 | 3.50% |
| Hyaluronic acid | 0.10% |
| Lipmax ™ | 10.00% |
| Pluronic ® | 5.00% |
| porpylene glycol | 5.00% |
| Durasoft ® | 1.00% |
| sodium carbonate | 11.00% |
| calcium carbonate | 22.00% |
| ethanol | 1.50% |
| benzyl OH | 1.00% |
| Total | 100.00% |

TABLE 2
Acylation Agent Treatment Formulation

| Chemical | |
|---|---|
| Component | Wt % |
| phospholipon 90G | 12.00% |
| EDTA | 0.24% |
| triethanolamine | 0.09% |
| tert-Amyl alcohol | 3.20% |
| triacetin | 43.70% - X % |
| isopropyl palmitate | 2.00% |
| Lipmax ™ | 24.00% |
| Pluronic ® | 12.00% |
| NaOH | 0.08% |
| Sodium Decanoate | 0.40% |
| Sodium Bicarbonate | 0.13% |
| Sodium Carbonate | 0.16% |
| benzyl OH | 2.00% |
| Glutaric Anhydride | X % |
| Total | 100.00% |

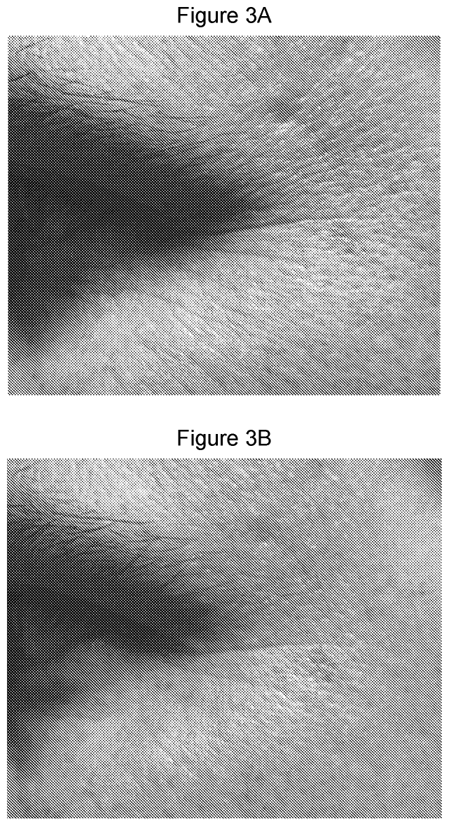

All treatment groups showed statistically significant increases in skin thickness versus control. On average, the control group saw a 0.03% decrease in thickness while the treatment groups saw the following thickness changes on average as shown with FIG. 3.

TABLE 3
Average Skin Thickness % change

| Glutaric | Skin thickness |
|---|---|
| Anhydride % | increase % |
| 1.67% | 8.00% |
| 3.33% | 12.16% |
| 5.00% | 9.11% |
| 6.67% | 6.00% |
| 10.00% | 3.81% |

Based on these results, the range of optimum dosing is likely between 3-4% glutaric anhydride.

### Example 2: Treatment and Pre-Treatment Time Optimization for Topically Delivered Glutaric Anhydride to Thicken Skin in an In Vitro Porcine Skin Model

In this experiment, glutaric anhydride in formulations of the invention in combination with a pre-treatment anhydrous buffering step in formulations of the invention were tested for its ability to thicken skin in an in vitro porcine skin model across a range of treatment and pre-treatment times.

In vitro tests were performed as follows: Porcine skin samples had their thickness measured, then were randomly sorted into treatment groups and given their respective treatments (described below). Trial design was built to assess these primary outcomes: change in skin thickness. Treatment groups randomized as follows:

a. Control—no treatment
b. 10 minutes of Anhydrous Buffering Treatment (Table 4) followed by 30 minutes of Acylation Agent Treatment (Table 5)
c. 20 minutes of Anhydrous Buffering Treatment (Table 4) followed by 30 minutes of Acylation Agent Treatment (Table 5)
d. 20 minutes of Anhydrous Buffering Treatment (Table 4) followed by 20 minutes of Acylation Agent Treatment (Table 5)

TABLE 4
Anhydrous Buffering Treatment Formulation

| Chemical | |
|---|---|
| Component | Wt % |
| cetyl OH | 2.00% |
| Myritol ® 312 | 8.00% |
| isododecane | 9.90% |
| Lipmax ™ | 4.00% |
| phospholipon 90G | 9.00% |
| Cetiol ® Ultimate | 7.00% |
| Tego ® 13-06 | 3.50% |
| Hyaluronic acid | 0.10% |
| Lip-Max | 10.00% |
| Pluronic ® | 5.00% |
| porpylene glycol | 5.00% |
| Durasoft ® | 1.00% |
| sodium carbonate | 11.00% |
| calcium carbonate | 22.00% |
| ethanol | 1.50% |
| benzyl OH | 1.00% |
| Total | 100.00% |

TABLE 5
Acylation Agent Treatment Formulation

| Chemical | |
|---|---|
| Component | Wt % |
| phospholipon 90G | 12.00% |
| EDTA | 0.24% |
| triethanolamine | 0.09% |
| tert-Amyl alcohol | 3.20% |
| triacetin | 39.70% |
| isopropyl palmitate | 2.00% |
| Lipmax ™ | 24.00% |
| Pluronic ® | 12.00% |
| NaOH | 0.08% |
| Sodium Decanoate | 0.40% |
| Sodium Bicarbonate | 0.13% |
| Sodium Carbonate | 0.16% |
| benzyl OH | 2.00% |
| Glutaric Anhydride | 4.00% |
| Total | 100.00% |

All treatment groups showed statistically significant increases in skin thickness versus control. On average, the control group saw a 0.05% decrease in thickness while the treatment groups saw the following thickness changes on average as shown with FIG. 6.

TABLE 6
Average Skin Thickness % change

| Timing Protocol | Skin thickness increase % |
|---|---|
| 10 min pre-treatment, | 14.33% |
| 30 min treatment | |
| 20 min pre-treatment, | 15.20% |
| 30 min treatment | |
| 20 min pre-treatment, | 9.24% |
| 20 min treatment | |

Based on these results, and the fact that pre-treatment times above 20 minutes or treatment times above 30 minutes started to show signs of skin irritation, we expect that the optimum protocol is 20 min of Anhydrous Buffering pre-treatment, followed by 30 min of Acylation Agent treatment

### Example 3: Use of Topically Delivered Acylation Agent for the Treatment of Glabellar Frown Lines

In this experiment, glutaric anhydride in formulations of the invention in combination with a pre-treatment anhydrous buffering step in formulations of the invention were tested for its ability to treat glabellar frown lines in comparison to injections of C. botulinum toxin.

In vivo tests were performed as follows: 34 Human subjects with glabellar frown lines were randomly sorted into treatment groups and given their respective treatments (described below). Subjects were assessed 30 days post-treatment. Trial design was built to assess these primary outcomes: subjective patient satisfaction. Treatment groups randomized as follows:

a. 10 to 12.5 units of C. Botulinum toxin per furrow
b. 20 minutes of Anhydrous Buffering Treatment (Table 7) followed by 30 minutes of Acylation Agent Treatment (Table 8)

TABLE 7
Anhydrous Buffering Treatment Formulation

| Chemical | |
|---|---|
| Component | Wt % |
| cetyl OH | 2.00% |
| Myritol ® 312 | 8.00% |
| isododecane | 9.90% |
| Lipmax ™ | 4.00% |
| phospholipon 90G | 9.00% |
| Cetiol ® Ultimate | 7.00% |
| Tego ® 13-06 | 3.50% |
| Hyaluronic acid | 0.10% |
| Lipmax ™ | 10.00% |
| Pluronic ® | 5.00% |
| porpylene glycol | 5.00% |
| Durasoft ® | 1.00% |
| sodium carbonate | 11.00% |
| calcium carbonate | 22.00% |
| ethanol | 1.50% |
| benzyl OH | 1.00% |
| Total | 100.00% |

TABLE 8
Acylation Agent Treatment Formulation

| Chemical | |
|---|---|
| Component | Wt % |
| phospholipon 90G | 12.00% |
| EDTA | 0.24% |
| triethanolamine | 0.09% |
| tert-Amyl alcohol | 3.20% |
| triacetin | 39.70% |
| isopropyl palmitate | 2.00% |
| Lipmax ™ | 24.00% |
| Pluronic ® | 12.00% |
| NaOH | 0.08% |
| Sodium Decanoate | 0.40% |
| Sodium Bicarbonate | 0.13% |
| Sodium Carbonate | 0.16% |
| benzyl OH | 2.00% |
| Glutaric Anhydride | 4.00% |
| Total | 100.00% |

All treatment groups showed statistically significant levels of patient satisfaction. On average the C. Botulinum Toxin group reported patient satisfaction scores of 5.18 on average (where 0 is no observed change and 7 was met highest expectations). The topical acylation agent treatment group reported patient satisfaction scores of 4.96 on average. There was no statistically significant difference between the two groups.

### Example 4—Use of Topically Delivered Acylation Agent for the Treatment of Crow's Feet Wrinkles

In this experiment, glutaric anhydride in formulations of the invention in combination with a pre-treatment anhydrous buffering step in formulations of the invention were tested for its ability to treat crow's feet wrinkles in comparison to injections of Onabotulinumtoxin A (ONA).

In vivo tests were performed as follows: 40 Human subjects with moderate to severe crow's feet wrinkles were randomly sorted into treatment groups and given their respective treatments (described below). Patients were assessed at baseline and 7, 30, and 120 days post treatment. Trial design was built to assess these primary outcomes: Duration of Effect, Degree of Improvement, and Patient Satisfaction. Treatment groups randomized as follows:

a. Right side of face: 10 to 15 units of Onabotulinumtoxin A; Left side of face: 20 minutes of Anhydrous Buffering Treatment (Table 9) followed by 30 minutes of Acylation Agent Treatment (Table 10)
b. Left side of face: 10 to 15 units of Onabotulinumtoxin A; Right side of face: 20 minutes of Anhydrous Buffering Treatment (Table 9) followed by 30 minutes of Acylation Agent Treatment (Table 10)

TABLE 9
Anhydrous Buffering Treatment Formulation

| Chemical | |
|---|---|
| Component | Wt % |
| cetyl OH | 2.00% |
| Myritol ® 312 | 8.00% |
| isododecane | 9.90% |
| Lipmax ™ | 4.00% |
| phospholipon 90G | 9.00% |
| Cetiol ® Ultimate | 7.00% |
| Tego ® 13-06 | 3.50% |
| Hyaluronic acid | 0.10% |
| Lipmax ™ | 10.00% |
| Pluronic ® | 5.00% |
| porpylene glycol | 5.00% |
| Durasoft ® | 1.00% |
| sodium carbonate | 11.00% |
| calcium carbonate | 22.00% |
| ethanol | 1.50% |
| benzyl OH | 1.00% |
| Total | 100.00% |

TABLE 10
Acylation Agent Treatment Formulation

| Chemical | |
|---|---|
| Component | Wt % |
| phospholipon 90G | 12.00% |
| EDTA | 0.24% |
| triethanolamine | 0.09% |
| tert-Amyl alcohol | 3.20% |
| triacetin | 39.70% |
| isopropyl palmitate | 2.00% |
| Lipmax ™ | 24.00% |
| Pluronic ® | 12.00% |
| NaOH | 0.08% |
| Sodium Decanoate | 0.40% |
| Sodium Bicarbonate | 0.13% |
| Sodium Carbonate | 0.16% |
| benzyl OH | 2.00% |
| Glutaric Anhydride | 4.00% |
| Total | 100.00% |

40% of crow's feet wrinkles treated by Onabotulinumtoxin A saw regression by day 120, compared to only 28% of wrinkles treated by the Acylation Agent. This difference was statistically significant.

Crow's feet wrinkles treated by Onabotulinumtoxin A saw peak improvement at the day 30 checkup—with average wrinkle severity scores (0—none, to 4—very severe) dropping from the baseline 3.20 to 1.60. Crow's feet wrinkles treated by the Acylation Agent saw peak improvement at the day 7 checkup—with average wrinkle severity scores (0—none, to 4—very severe) dropping from the baseline 3.20 to 1.80.

All treatment groups showed statistically significant levels of patient satisfaction. On average, when rating the Onabotulinumtoxin A side of the face, patients reported satisfaction scores of 2.00 on average (where 0 is no improvement and 3 was very good improvement). The topical acylation agent treatment side had patient satisfaction scores of 2.12 on average. There was no statistically significant difference between the two groups.

### Example 5: Use of Topically Delivered Acylation Agent for the Treatment of Peri-Orbital Skin Texture Roughness

In this experiment, glutaric anhydride in formulations of the invention in combination with a pre-treatment anhydrous buffering step in formulations of the invention were tested for its ability to treat peri-orbital skin texture roughness in comparison to injections of C. botulinum toxin type A.

In vivo tests were performed as follows: 40 Human subjects with per-orbital wrinkles were randomly sorted into treatment groups and given their respective treatments (described below). Trial design was built to assess these primary outcomes: skin texture roughness. Treatment groups randomized as follows:

a. 12 units of C. Botulinum toxin A per peri-orbital region
b. 20 minutes of Anhydrous Buffering Treatment (Table 11) followed by 30 minutes of Acylation Agent Treatment (Table 12)

TABLE 11
Anhydrous Buffering Treatment Formulation

| Chemical | |
|---|---|
| Component | Wt % |
| cetyl OH | 2.00% |
| Myritol ® 312 | 8.00% |
| isododecane | 9.90% |
| Lip-Max | 4.00% |
| phospholipon 90G | 9.00% |
| Cetiol ® Ultimate | 7.00% |
| Tego ® 13-06 | 3.50% |
| Hyaluronic acid | 0.10% |
| Lipmax ™ | 10.00% |
| Pluronic | 5.00% |
| porpylene glycol | 5.00% |
| Durasoft ® | 1.00% |
| sodium carbonate | 11.00% |
| calcium carbonate | 22.00% |
| ethanol | 1.50% |
| benzyl OH | 1.00% |
| Total | 100.00% |

TABLE 12
Acylation Agent Treatment Formulation

| Chemical | |
|---|---|
| Component | Wt % |
| phospholipon 90G | 12.00% |
| EDTA | 0.24% |
| triethanolamine | 0.09% |
| tert-Amyl alcohol | 3.20% |
| triacetin | 39.70% |
| isopropyl palmitate | 2.00% |
| Lipmax ™ | 24.00% |
| Pluronic | 12.00% |
| NaOH | 0.08% |
| Sodium Decanoate | 0.40% |
| Sodium Bicarbonate | 0.13% |
| Sodium Carbonate | 0.16% |
| benzyl OH | 2.00% |
| Glutaric Anhydride | 4.00% |
| Total | 100.00% |

All treatment groups showed statistically significant levels of improvement in skin texture roughness (STR). On average the C. Botulinum Toxin A group saw a significant decrease of STR of 17.08% at a 4 week follow up. The topical acylation agent treatment group saw a significant decrease of STR of 19.82% at a 4 week follow up. There was no statistically significant difference between the two groups.

### Example 6: Use of Topically Delivered Acylation Agent for the Treatment of Crow's Feet Wrinkles in Comparison to Multiple Botulinum Toxin Based Treatment Protocols

In this experiment, glutaric anhydride in formulations of the invention in combination with a pre-treatment anhydrous buffering step in formulations of the invention were tested for its ability to treat crow's feet wrinkles in comparison to intramuscular injections, intradermal microdroplets, and nanomicroneedles of C. botulinum toxin A.

In vivo tests were performed as follows: 40 Human subjects with moderate to severe crow's feet wrinkles were randomly sorted into treatment groups and given their respective treatments (described below). Outcomes were assessed 4 weeks post-treatment. Trial design was built to assess these primary outcomes: blinded physician-rated crow's feet severity on a 0 to 4 scale. Treatment groups randomized as follows:

a. Intramuscular injection of 14 units of C. Botulinum toxin A (7 per side)
b. Intradermal microdroplet injection of 14 units of C. Botulinum toxin A (7 per side)
c. Nanomicroneedle injection of 14 units of C. Botulinum toxin A (7 per side)
d. 20 minutes of Anhydrous Buffering Treatment (Table 13) followed by 30 minutes of Acylation Agent Treatment (Table 14) on each side

TABLE 13
Anhydrous Buffering Treatment Formulation

| Chemical | |
|---|---|
| Component | Wt % |
| cetyl OH | 2.00% |
| Myritol ® 312 | 8.00% |
| isododecane | 9.90% |
| Lipmax ™ | 4.00% |
| phospholipon 90G | 9.00% |
| Cetiol ® Ultimate | 7.00% |
| Tego ® 13-06 | 3.50% |
| Hyaluronic acid | 0.10% |
| Lip-Max | 10.00% |
| Pluronic | 5.00% |
| porpylene glycol | 5.00% |
| Durasoft ® | 1.00% |
| sodium carbonate | 11.00% |
| calcium carbonate | 22.00% |
| ethanol | 1.50% |
| benzyl OH | 1.00% |
| Total | 100.00% |

TABLE 14
Acylation Agent Treatment Formulation

| Chemical | |
|---|---|
| Component | Wt % |
| phospholipon 90G | 12.00% |
| EDTA | 0.24% |
| triethanolamine | 0.09% |
| tert-Amyl alcohol | 3.20% |
| triacetin | 39.70% |
| isopropyl palmitate | 2.00% |
| Lipmax ™ | 24.00% |
| Pluronic | 12.00% |
| NaOH | 0.08% |
| Sodium Decanoate | 0.40% |
| Sodium Bicarbonate | 0.13% |
| Sodium Carbonate | 0.16% |
| benzyl OH | 2.00% |
| Glutaric Anhydride | 4.00% |
| Total | 100.00% |

All treatment groups showed statistically significant levels of wrinkle improvement. All group's baseline wrinkle assessment was between 3.50-3.60 on average. Post treatment, the intramuscular injection group improved to 0.50 on average, compared to 0.60 for intradermal microdroplet injections and 1.65 for nano micro needles. The topically applied acylation agent improved to an average score of 0.67.

In some embodiments, the formulation for transdermal delivery of a buffering agent through the skin of a subject, comprises a formulation of Table 1, Table 2, Table 4, Table 5, Table 7, Table 8, Table 9, Table 10, Table 11, Table 12, Table 13, or Table 14.

In closing, regarding the exemplary embodiments of the present invention as shown and described herein, it will be appreciated that the formulations disclosed herein are configured for buffering therapy with or without an additional therapeutic agent. Because the principles of the invention may be practiced in a number of configurations beyond those shown and described, it is to be understood that the invention is not in any way limited by the exemplary embodiments but is generally directed to a transdermal formulation and is able to take numerous forms to do so without departing from the spirit and scope of the invention. It will also be appreciated by those skilled in the art that the present invention is not limited to the particular components disclosed but may instead entail other functionally comparable formulation components, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc. —for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A pre-treatment and/or treatment formulation for transdermal delivery of a dermal pre-treatment agent and/or one or more dermal contouring agents through the skin of a subject,
wherein the pre-treatment formulation comprises:
a) a buffering agent comprising at least one carbonate salt, lysine, tris, a phosphate buffer and/or 2-imidazole-1-yl-3-ethoxycarbonylpropionic acid (TEPA), or a combination thereof in an amount between about 0.05-60% w/w; and
b) a pre-treatment penetrant portion in an amount between about 40 to 99.95% w/w, and
wherein the treatment formulation comprises:
c) one or more acylating agents in an amount between about 0.25-25% w/w; and
d) a treatment penetrant portion in an amount between about 40 to 75% w/w.

2. The formulation of claim 1, wherein the acylating agent is selected from the group consisting of glutaric anhydride, EDTA anhydride, octenyl-succinic anhydride, maleic anhydride, succinic anhydride, citraconic anhydride, methyl succinic anhydride, itaconic anhydride, methyl glutaric anhydride, dimethyl glutaric anhydride, phthalic anhydride, oxalyl chloride, malonyl chloride, (chlorosulfonyl)acetylchloride, (chlorosulfonyl)benzoic acid, 4-chloro-3-(chlorosulfonyl)-5-nitroebnzoicacid, 3-(chlorosulfonyl)-p-anisic acid, 3-(sulfonyl)benzoic acid, 3,5-Dimethoxybenzoyl chloride, acetic anhydride, chloroacetic anhydride, propionic anhydride, butyric anhydride, isobutyric anhydride, isovaleric anhydride, hexanoic anhydride, acetylchloride, propionylchloride, dichloropropionylchloride, butyryl chloride, isobutyryl chloride, valeryl chloride, ethanesulfonyl chloride, methanesulfonyl chloride, 1-butanesulfonyl chloride, 4,6-diamino-2-methylthiopyrimidine-5-Sulfonic acid, and combinations thereof.

3. The formulation of claim 1, wherein the pre-treatment formulation is anhydrous.

4. The formulation of claim 1, wherein the pre-treatment and/or treatment penetrant penetrant portion comprise lecithin organogel in an amount between about 10-70% w/w of the formulation.

5. The formulation of claim 1, wherein the pre-treatment and/or treatment formulation comprises less than about 35% w/w lecithin organogel.

6. The formulation of claim 1, wherein the pre-treatment and/or treatment formulation comprises less than about 12% w/w lecithin organogel.

7. The formulation of claim 1, wherein the pre-treatment and/or treatment penetrant portion further comprises a detergent portion in an amount between about 1 to 70% w/w.

8. The formulation of claim 7, wherein the detergent portion comprises a nonionic surfactant in an amount between about 2-25% w/w of the penetrant portion; and a polar solvent in an amount less than 5% w/w of the penetrant portion.

9. The formulation of claim 1, wherein the buffering agent is in an amount between about 0.05-36% w/w of the formulation.

10. The formulation of claim 1, wherein the pre-treatment and/or treatment penetrant portion is in an amount between about 44-80% w/w of the formulation.

11. The formulation of claim 1, wherein the pre-treatment and/or treatment penetrant portion comprises an alcohol in an amount less than 10% w/w of the formulation.

12. The formulation of claim 1, wherein the pre-treatment and/or treatment penetrant portion comprises lecithin organogel, an alcohol, a surfactant, and a polar solvent.

13. The formulation of claim 1, wherein the pre-treatment and/or treatment penetrant portion comprises a mixture of xanthan gum, lecithin, sclerotium gum, pullulan, or a combination thereof in an amount less than 5% w/w of the formulation.

14. The formulation of claim 1, wherein the pre-treatment and/or treatment penetrant portion comprises a mixture of caprylic triglycerides and capric triglycerides in amount less than 8% w/w of the formulation.

15. The formulation of claim 1, wherein the pre-treatment and/or treatment penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 12% w/w of the formulation.

16. The formulation of claim 1, wherein the pre-treatment and/or treatment penetrant portion comprises cetyl alcohol in amount less than 5% w/w of the formulation.

17. The formulation of claim 1 wherein the pre-treatment and/or treatment penetrant portion comprises stearic acid in an amount less than 5% w/w of the formulation.

18. The formulation of claim 1, wherein the pre-treatment and/or treatment formulation comprises a gelling agent in an amount less than 5% w/w of the formulation.

19. The formulation of claim 1, wherein the carbonate salt is sodium bicarbonate milled to a particle size less than 70 μm, wherein the sodium bicarbonate is solubilized in the formulation in an amount less than 10% w/w of the formulation.

20. The formulation of claim 1, wherein the pre-treatment and/or treatment formulation each independently further comprise tranexamic acid in an amount less than 5% w/w of the formulation.

21. The formulation of claim 1, wherein the pre-treatment and/or treatment formulation each independently further comprise a polar solvent in an amount less than 5% w/w of the formulation.

22. The formulation of claim 1, wherein the pre-treatment and/or treatment formulation each independently further comprise a humectant, an emulsifier, an emollient, or a combination thereof.

23. The formulation of claim 1, wherein the pre-treatment formulation has a pH of 7-10.5.

24. The formulation of claim 1, wherein the pre-treatment formulation comprises:
Isododecane 1-20% w/w;
Tego 13-06 1-10% w/w;
Hyaluronic acid 0.05-10% w/w;
Propylene glycol in an amount between about 0.5-15% w/w;
Sodium carbonate in an amount between about 1-25% w/w;
Calcium carbonate in an amount between about 1-36% w/w.
Ethanol in an amount between about 0.5-10% w/w; and
Benzyl alcohol in an amount between about 0.25-5% w/w.
The formulation of claim 1, wherein the treatment formulation comprises:
EDTA (Disodium ethylenediaminetetraacetate dihydrate) in an amount between about 0.05-5% w/w;
Triethanolamine in an amount between about 0.05-2% w/w;
tert-Amyl alcohol in an amount between about 0.5-10% w/w;
Triacetin in an amount between about 1-60% w/w;
Isopropyl palmitate in an amount between about 1-10% w/w;
Sodium hydroxide in an amount between about 0.005-2% w/w;
Sodium decanoate in an amount between about 0.05-5% w/w;
Sodium bicarbonate in an amount between about 0.05-10% w/w;
Sodium carbonate in an amount between about 0.05-10% w/w;
Benzyl alcohol in an amount between about 0.25-5% w/w; and
Glutaric anhydride in an amount between about 1-10% w/w

25. A pre-treatment and/or treatment formulation for transdermal delivery of a dermal pre-treatment agent and/or one or more dermal contouring agents through the skin of a subject,
wherein the pre-treatment formulation comprises: Table 1, Table 4, Table 7, Table 9, Table 11, or Table 13; and
wherein the treatment formulation comprises: Table 2, Table 5, Table 8, Table 10, Table 12, or Table 14.

26. A method for transdermal delivery of the pre-treatment and/or treatment formulation of claim 1, through the skin of a subject.

27. The method of claim 26, wherein the pre-treatment formulation is applied to a subject and the treatment formulation is applied to the subject after the pre-treatment formulation.

28. A method to expand tissue volume comprising applying the pre-treatment and/or treatment formulation of claim 1, through the skin of a subject, the method comprising:
a) mixing the one or more acylating agents in powdered form with the treatment penetration portioning immediately followed by application of the mixture to a skin area of the subject.

29. A method to expand tissue volume comprising applying the pre-treatment and/or treatment formulation of claim 1, through the skin of a subject, the method comprising:
a) mixing the one or more acylating agents in powdered form with the treatment penetration portioning immediately followed by application of the mixture to a skin area of the subject; or
b) first, applying to a skin area of the subject the pre-treatment formulation buffered at pH 7-11.5 comprising an effective amount of said acylating agent, 25-70% w/w lecithin organogel, 1-20% w/w benzyl alcohol, and a nonionic detergent in the presence of a bile salt; and second mixing the one or more acylating agents in powdered form with the treatment penetration portioning immediately followed by application of the mixture to a skin area of the subject.

30. A method to expand volume and maintain hydration of tissue which method comprises applying to an area the pre-treatment and/or treatment formulation of claim 1, through the skin of a subject, the method comprising:
a) mixing the one or more acylating agents in powdered form with the treatment penetration portioning immediately followed by application of the mixture to a skin area of the subject.

31. A method to expand volume and maintain hydration of tissue which method comprises applying to an area the pre-treatment and/or treatment formulation of claim 1, through the skin of a subject, the method comprising:
a) mixing the one or more acylating agents in powdered form with the treatment penetration portioning immediately followed by application of the mixture to a skin area of the subject; or
b) first, applying to a skin area of the subject the pre-treatment formulation buffered at pH 7-11.5 comprising an effective amount of said acylating agent, 25-70% w/w lecithin organogel, 1-20% w/w benzyl alcohol, and a nonionic detergent in the presence of a bile salt; and second mixing the one or more acylating agents in powdered form with the treatment penetration portioning immediately followed by application of the mixture to a skin area of the subject.

32. A method to restore the barrier effect of skin after transdermal treatment, which method comprises applying the pre-treatment and/or treatment formulation of claim 1, through the skin of a subject, the method comprising:
a) mixing the one or more acylating agents in powdered form with the treatment penetration portioning immediately followed by application of the mixture to a skin area of the subject.

33. A method to restore the barrier effect of skin after transdermal treatment, which method comprises applying the pre-treatment and/or treatment formulation of claim 1, through the skin of a subject, the method comprising:
a) mixing the one or more acylating agents in powdered form with the treatment penetration portioning immediately followed by application of the mixture to a skin area of the subject; or
b) first, applying to a skin area of the subject the pre-treatment formulation buffered at pH 7-11.5 comprising an effective amount of said acylating agent, 25-70% w/w lecithin organogel, 1-20% w/w benzyl alcohol, and a nonionic detergent in the presence of a bile salt; and second mixing the one or more acylating agents in powdered form with the treatment penetration portioning immediately followed by application of the mixture to a skin area of the subject.
